(19) United States
(12) Patent Application Publication
(10) Pub. No.: US 2009/0093852 A1
(43) Pub. Date: Apr. 9, 2009

(54) SPINAL STABILIZATION TREATMENT METHODS FOR MAINTAINING AXIAL SPINE HEIGHT AND SAGITAL PLANE SPINE BALANCE

(76) Inventor: Richard A. Hynes, Melbourne Beach, FL (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(21) Appl. No.: 12/245,811

(22) Filed: Oct. 6, 2008

Related U.S. Application Data
(60) Provisional application No. 60/977,667, filed on Oct. 5, 2007.

(51) Int. Cl. A61B 17/70

(52) U.S. Cl. 606/86 A; 606/246

(57) ABSTRACT

A proactive spinal treatment method is for maintaining axial spine height and sagital plane spine balance in a spine comprising vertebrae and intervertebral discs between adjacent vertebrae. The method may include collecting a plurality of spinal health parameters relating to predicted spinal degeneration risk for a given patient's spine, and analyzing the plurality of spinal health parameters to generate a proposed stabilizing implantation treatment using a finite element spinal model. The method may further include performing the proposed stabilizing implantation treatment on the given patient's spine to proactively treat the patient to maintain axial spine height and sagital plane spine balance.

### CROSS-REFERENCE TO RELATED APPLICATIONS

This application claims the benefit of U.S. Provisional Application No. 60/977,667, filed Oct. 5, 2007, which is hereby incorporated herein in its entirety by reference.

### FIELD OF THE INVENTION

The present invention relates to the field of musculoskeletal treatment methods, and, more particularly, to spinal treatment methods.

### BACKGROUND OF THE INVENTION

Musculoskeletal conditions can be painful and debilitating for any patient, but especially so for elderly patients. Not only is pain a significant issue, but loss of central balance of the spinal axis, loss of axial height and compression of the major organ cavities, the chest and abdomen may lead to poor medical outcomes. One bone disease that commonly affects the elderly is osteoporosis. Osteoporosis causes the density and micro-architecture of bones to be degraded. The result is that bones become more susceptible to osteoporotic fractures, which occur under slight amounts of stresses that would not typically cause fractures in a normal (i.e., healthy) bone. Bones which are particularly susceptible to osteoporotic fractures include those in the vertebral column, hip and wrist.

In particular, collapse of a vertebra from compression fractures can result in problems such as a hunched forward or bent stature (kyphosis), bent spine (scoliosis), loss of axial height, loss of sagital plane balance and reduced mobility. Moreover, vertebral collapse can be especially problematic because this can impinge upon nerves in the spinal cord, which may result in numbness, acute back pain, cardiopulmonary disorders, abdominal disorders and potentially other medical disorders. The kyphosis also places the balance of the head and position of the vestibular apparatus in the ear, anterior to the central axis and may contribute to the ever increasing falls of the elderly. These falls lead to hip and wrist fractures as well.

Various approaches have been developed for treating bones, such as vertebrae, which have previously suffered a fracture. One such example is set forth in U.S. Published Patent Application No. 2006/0106459, which discloses a system for treating an abnormal vertebral body, such as one with a compression fracture. The system includes a biocompatible flow-through implant structure configured with a three-dimensional interior web that defines flow openings therein for cooperating with a two-part hardenable bone cement. The flow-through structure is capable of compacted and extended shapes and in one embodiment provides gradient inflow openings for controlling flow parameters of a bone cement injected under high pressure into the interior thereof.

Other approaches have been developed for treating damaged or collapsed vertebral discs, which may also cause one or more of the problems discussed above. Such techniques typically involve the injection of bone cements and other agents to provide a total or partial vertebral body or disc replacement, which is commonly referred to as vertebroplasty. One example is set forth in U.S. Published Patent Application No. 2002/0045942, which discloses techniques and compositions for repairing a damaged intervertebral disc. A biologically inert thermoplastic elastomer precursor is introduced through the annulus fibrosus and into the nucleus pulposus in a liquid state and with sufficient pressure to re-inflate the damaged disc to its normal undamaged dimensions. Thereafter, the thermoplastic elastomer precursor is cured in situ to a hardness sufficient to support normal postural compressive loads and prevent the disc from returning to its damaged dimensions. This is done with a syringe including a barrel filled with the liquid thermoplastic elastomer precursor, an operating plunger, and a projecting needle that is positioned adjacent the damaged disc. The needle inserted through the annulus fibrosus and into the nucleus pulposus, and the plunger is operated to inject the liquid thermoplastic elastomer precursor into the nucleus pulposus.

Another related technique is referred to as balloon-assisted vertebroplasty. By way of example, U.S. Pat. No. 6,958,077 discloses an inflatable nuclear prosthesis method in which the nucleus of an intervertebral disc is replaced with a construct including a distendable balloon sack that is inflated with a hardenable material. The balloon is detached in situ when the injected material has hardened.

Various approaches for analyzing musculoskeletal problems have also been created. One example is set forth in U.S. Patent Pub. No. 2007/0093998, which discloses a method for biomechanically simulating a set of osseous joints. The method includes recording a digital three dimensional model embodied at least partially in the form of rigid bodies interconnected by joints in a reference position, personalizing the model geometry by specific data of the patient in the reference position, and personalizing the digital model by particulating interaction parameters of each joint connecting the rigid bodies according to detected patient characteristics. The particularization of the interaction parameters includes obtaining the space position of at least the part of the rigid bodies, interpolating for determining the calculated position of other rigid bodies to produce a numerical index containing the relative position of each rigid body, performing at least one defined constraint on the patient and collecting information on the general balance position of the patient, and determining analytical functions which make it possible to approximate the interaction parameters, and thereby reproduce the measured relative positions for each couple of rigid bodies.

Despite the potential benefits of such analysis and treatment procedures in certain circumstances, additional treatment methods may be desirable in many applications.

### SUMMARY OF THE INVENTION

In view of the foregoing background, it is therefore an object of the present invention to provide methods for maintaining axial spine height and sagital plane spine balance, for example.

This and other objects, features, and advantages are provided by a proactive spinal treatment method for maintaining axial spine height and sagital plane spine balance in a spine comprising vertebrae and intervertebral discs between adjacent vertebrae. The method may include collecting a plurality of spinal health parameters relating to predicted spinal degeneration risk for a given patient's spine, and analyzing the plurality of spinal health parameters to generate a proposed stabilizing implantation treatment using a finite element spinal model. The method may further include performing the proposed stabilizing implantation treatment on the given patient's spine to proactively treat the patient to maintain axial spine height and sagital plane spine balance.

Performing the proposed stabilizing implantation treatment may include implanting at least one stabilizer adjacent a plurality of spaced apart locations along the spine. By way of example, the plurality of spinal health parameters are selected from a group including patient age, medical comorbidities, family history, and patient fracture history. Additionally, the plurality of spinal health parameters may be selected from a group comprising x-ray, dual energy x-ray absorptiometry (DEXA) scan results, magnetic resonance imaging scan results, and computerized axial tomography scan results, for example.

Furthermore, performing the proposed stabilizing implantation treatment may include implanting a plurality of opposing magnetic elements within the given patient's spine. By way of example, implanting the plurality of opposing magnetic elements may include implanting opposing regions of polymethylmethacrylate (PMMA) comprising magnetic particles of opposite polarity. In some embodiments, performing the proposed stabilizing implantation treatment may include implanting at least one metallic element between an opposing pair of vertebrae and inducing a magnetic field for causing the at least one metallic element to space apart the pair of vertebrae.

In addition, the method may also include performing a spinal elongation procedure to elongate the given patient's spine to an elongated state longer before performing the proposed stabilizing implantation treatment. For example, the spinal elongation procedure may include at least one of traction, bracing, suspension, inversion, and chiropractic manipulation.

### BRIEF DESCRIPTION OF THE DRAWINGS

FIG. 1 is a flow diagram illustrating a proactive spinal treatment method for maintaining axial spine height and sagital plane spine balance in accordance with one aspect of the invention.

FIG. 2 is an anterior view of a spine demonstrating stabilizing implants at a plurality of locations for maintaining axial spine height and sagital plane spine balance.

FIG. 3 is a flow diagram illustrating additional proactive spinal treatment method aspects in accordance with the invention.

FIGS. 4 and 5 are side views of the spine of FIG. 3 demonstrating various stabilizing implants that may be used in accordance with the invention.

FIGS. 6 and 7 are anterior and side views, respectively, of an exemplary system for thoracic spine stabilization that may be used in accordance with the proactive spinal treatment methods of the invention.

### DETAILED DESCRIPTION OF THE EMBODIMENTS

The present invention will now be described more fully hereinafter with reference to the accompanying drawings, in which preferred embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout, and prime notation is used to indicate similar elements in alternate embodiments.

Generally speaking, methods for bone, disc, ligaments, muscle, fascia, facet joints, hip and wrist, etc., stabilization/deformity prevention are described herein. While the methods described herein may be used for numerous bones, soft tissues and joints, it is particularly advantageous for spinal deformity prevention uniquely applied to loss of axial height and kyphosis but not exclusive of other deformities. The approach includes, in part, prophylactic and preventative injection of stabilizing materials into the cervical, thoracic and/or lumbar spine for vertebral disc and bone stabilization. This procedure may advantageously keep a patient's spine from losing significant height as the patient ages by preventing the kyphosis or bending that comes with age from disc degeneration and/or bone fracture and eventual collapse of the vertebral bodies. Preventative injection of disc and other tension banding of soft tissue structures may help prevent loss of height of each individual disc. Such bending of the spine is typically associated with the hunched over or “old age” appearance of many elderly patients.

Maintenance of axial height can be difficult as aging occurs. Loss of height is due primarily to the loss of space between the vertebral bones, fracture of the vertebral bones, bending (kyphosis), and sagital and coronal plane imbalance over primarily the thoracic and lumbar spine, but the cervical spine region may contribute as well. Despite the increasing aging population, typical surgical and medical treatment methods do not attempt to maintain the axial height and balance of the patient nor the spine, i.e., to prevent the loss of axial height and balance before it occurs. Instead, most approaches attempt to reactively fix or correct fractured bones of the spine or correct kyphosis after it occurs, but unfortunately this often requires invasive surgery that can be complex and costly, and is particularly undesirable to perform on an elderly patient who may be susceptible to longer and more difficult recoveries as well as greater risks for complications during surgery.

There is also a paucity of approaches to address the problem of the medically induced longevity and life expectancy of patients which, on one hand, extends the cardiopulmonary function and yet simultaneously creates certain failure of the spine due to the extended length of life itself. The spine has substantial failure in the bone and soft tissue by the early 70's in a significant percentage of the population. The spine longevity has not kept up with the medical longevity and hence we now have a dramatic problem evolving with respect to quality of life. It does the patient little benefit to maintain medical health into the 90's and 100's if the spine collapses and fails in the 70's and 80's.

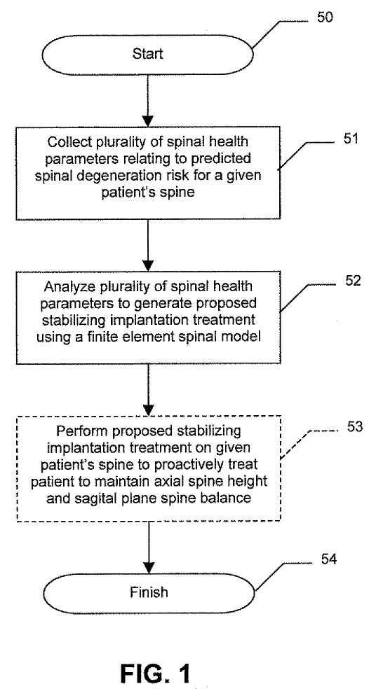

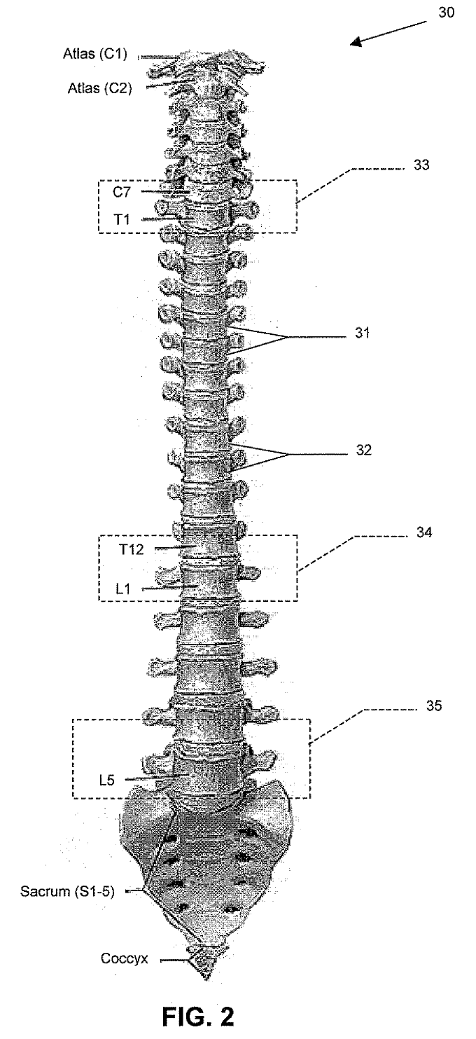

In accordance with one aspect, a proactive spinal treatment method for maintaining axial spine height and sagital plane spine balance in a spine 30 comprising vertebrae 31 and intervertebral discs 32 between adjacent vertebrae is now described with reference to FIGS. 1 and 2. The method begins (Block 50) with collecting a plurality of spinal health parameters relating to predicted spinal degeneration risk for a given patient's spine. As will be discussed further below, exemplary spinal health parameters may include medical history parameters such as patient age, medical comorbidities, family history, patient fracture history, etc. Moreover, other exemplary spinal health parameters that may be considered are physical parameters collected from a physical examination or scan of the patient. By way of example, such physical parameters may include dual energy x-ray absorptiometry (DEXA) scan results, magnetic resonance imaging scan results, computerized axial tomography scan results, etc., as will also be discussed further below. It should be noted that various combinations of these (and other) parameters may be used in different embodiments, and that not all parameters may be required in certain embodiments, as will be appreciated by those skilled in the art.

Once the appropriate parameters are collected, the parameters are then analyzed to generate a proposed stabilizing implantation treatment using a computer model, such as a finite element spinal model, for example, at Block 52. As will be appreciated by those skilled in the art, a finite element analysis model is a computer model of system that can be stressed and analyzed for determining how the system reacts to stresses, and what its failure points are. With respect to structural failures, finite element analysis may be used to help determine design or system modifications to avoid such failures. A finite element analysis model includes a plurality of nodes which form a grid called a mesh. As discussed in an article by Widas entitled “Introduction to Finite Element Analysis” published in April 2007 at http://www.sv.vt.edu/classes/MSE2094_NoteBook/97ClassProj/num/widas/history.html, the mesh is programmed to include the material and structural properties that determine how the structure will react to various loading conditions. Nodes are assigned at a different densities throughout the material based upon on the anticipated stress levels of a particular area. Regions which receive large amounts of stress usually have a higher node density than those which experience little or no stress.

While some finite element analysis models have been developed for modeling spinal systems, they are usually implemented on a relatively small scale (i.e., a few vertebrae), or are typically used for the purpose of determining the effect of placing an implant device at a given location in a patient in a reactive fashion to correct a previously existing condition. As noted above, the present approach may advantageously utilize a finite element analysis model to not only predict the likelihood of risk to a patient of spinal deformities or fracture (and thus spinal height loss and/or imbalance) based upon his or her particular parameters, but also to provide a prospective or prophylactic treatment regimen to help avoid such deformities.

One exemplary approach for generating a finite element analysis model of a normal spine is to first perform a Computerized Axial Tomographic (CAT) scan of spine, which will generate points that define surfaces of the various spinal components to be included in the model. A normal, healthy spine may be used as the baseline for the model, which upon completion may then be modified using parameters from individual patients to determine how such parameters will affect the spine, and determine the appropriate types and locations for implants to help prevent the occurrence of likely deformities or fractures.

Upon using the CAT scan to determine the appropriate surface points, a 3D (or 2D in some embodiments) computer model (e.g., a computer aided design (CAD)) of the spine is created. This may be generated automatically with the appropriate software application, or manually. Volumes may then be added to the structures with the CAD model, which again may be added in an automated fashion with the appropriate software application, or manually. A finite element model may then be generated, again using an appropriate finite element software application, using the volumes previously generated. That is, the CAD model may be imported inside a finite element software package. An analyst may then assign to the various components of the spine their own respective material values, such as elasticity modulus, etc. By way of example, ANSYS, Inc. of Canonsburg, Pa. provides various mechanical simulation software packages that may be used for generating and analyzing a spinal finite element model, as will be appreciated by those skilled in the art, although other suitable simulation tools may also be used.

The finite element model may be customized to different levels depending upon the sophistication of the analysis that is desired. For example, each disc may have its own values, which may be dependent upon the three spatial directions. For example, the body of the vertebrae has different material values than the spinous processes. If a more thorough analysis is required, these individual material values may be used, although in some embodiments a single approximation value could be used for simplicity. Another factor that may be included is friction coefficients that may be assigned between vertebrae and discs. In addition, “cables” may be created that simulate tendons between vertebrae and between vertebrae and discs, which also may be assigned respective material values. The model may then be “solved” using the finite element software.

Generally speaking, the more nodes and material differences that are included in the model, the more accurate the resulting analysis will be, but the complexity of the model and thus the time to create and process (both human and computer time) the model will increase accordingly. As such, the complexity of the given model may be balanced in a given application with the level of analysis and the various parameters that need to be analyzed for patients, as will be appreciated by those skilled in the art.

The use of such a finite element analysis to determine an appropriate spinal stabilizing implantation treatment will be discussed further below. Generally speaking, the treatment is selected and performed on the given patient's spine to proactively treat the patient to maintain axial spine height and sagital plane spine balance, at Block 53, thus concluding the method illustrated in FIG. 1.

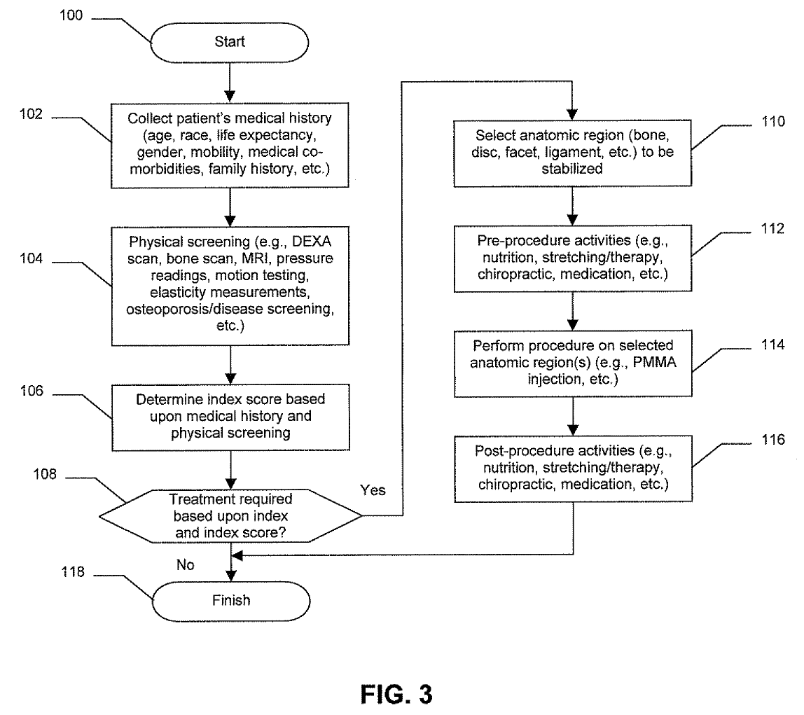

Referring now to FIG. 3, the present approach involves a proactive and/or preventative treatment method designed to match the ever-increasing longevity of patient's medical and structural potential, as opposed to a reactive surgical correction of a medical condition and a worsening gap between medical and structural longevity. A treatment strategy for relatively less invasive fixation of the anatomical components of the spine (or other bones/regions) is used, with relatively minimal risk in an aging spine population that is not able to tolerate invasive procedures well. This treatment strategy may narrow the gap that currently exists between the medical longevity and structural longevity. This is particularly important as medical solutions are advancing at a rapid rate without any current method for structural longevity improvements. Combinations of materials and anatomic regions are selected that will maintain the axial spine balance and height as a patient continues to age.

Various other potential benefits of this approach include, but are not limited to, cosmetic (i.e., the patient is less likely to suffer from a hunched-over “old age” appearance), medical, and physiological benefits with improvement in the mobility of the patient. Moreover, this approach may also advantageously mitigate against medical conditions that can otherwise occur from spinal deformation (e.g., cardiopulmonary disease, pain, numbness, etc.), and therefore potentially decrease disabilities, medical care costs, and hospital admissions.

Local and sometimes general anesthesia may be used for the stabilizing implant procedure. Generally speaking, the particular anatomical structures, materials, location of treatment areas, and length of procedure will be specific to each patient based upon factors such as age, gender, race, comorbidities, etc. Each patient may have a specific method and strategy applied by pre-procedure statistical analysis of risk, history and condition upon presentation. The stabilizing agents/inserts work together to help prevent fractures of the spine, collapse of the disc, facets and/or ligaments, and maintain sagital balance.

The specific aspects of applying treatment to each patient's condition may be based in a model that is predictive, accurate and reproducible, such as the finite element model discussed above. Using current knowledge of genetic heritage, gender differences, and medical comorbitites as well as statistical risk analysis to identify “at risk” patients and bony anatomical structures, one can delineate a more refined or specific method of treatment for each patient which further mitigates against unnecessary medical or surgical treatment. For example, combining the statistical risk of a thoracic T12 vertebral compression fracture (VCF) (see FIG. 2) with a patient's age, DEXA score, medical comorbidity score, family history, patient's past fracture history, life expectancy, etc., may be used to determine the need for preventative intervention from a procedural standpoint.

In accordance with one example a point system from one to five (or other scale) may be used to weigh each variable and determine the “at risk” levels which would benefit from preventative procedural intervention and which would not need intervention. Such model may also be also used for consideration of the disc and other potential soft tissue organs “at risk” for failure. Of course, the variables may be different between bone and soft tissue considerations. Furthermore, a composite model which assigns a consolidated “at risk” score may be created which not only determines bone and soft tissue at risk structures in need of preventative procedural treatment, from an individual perspective, but also a predictive model of loss of axial height and sagital plane imbalance to identify individuals who would benefit from a more extensive bone and soft tissue approach to prevent the untoward consequences of collapse and deformity of the spine. Again, this analysis may be generated based upon the above-noted normal spinal finite element model, and changing the parameters thereof with those specific to a given patient, as will be appreciated by those skilled in the art.

With respect to spinal implementations, various combinations of treatment options may be used including, vertebral bone alone; disc alone; vertebral bone and disc; vertebral bone, disc and facet joint; vertebral bone, disc, facet joint and ligament (s) (which may include, for example, the anterior longitudinal, posterior longitudinal, intra and supra spinous ligaments, and facet capsule ligaments). This may include lamina bone with pars and spinous process, as well as the muscles of the posterior spine, lateral and anterior spine.

The procedure may advantageously be used for balancing the modulus of elasticity, pressure and tension of the spine, coronal and sagital plane balance of the spine, and motion of the spine, by statistical analysis to provide selected materials in desired combinations and anatomic coupling to provide a relatively less invasive procedure with respect to certain typical post-treatment procedures, for example. The pre-procedure testing that may be used to determine the appropriate treatment methodology may include a DEXA scan, bone scan, MRI, CT scan, as well as other similar tests that help determine the actual quality, strength and ability of the anatomical structures to maintain the axial height based upon pressure readings, motion testing, disc and bone pressures, elasticity measurements of ligaments, osteoporosis and other medical factors, as will be appreciated by those skilled in the art. In some applications, the procedures may advantageously be performed though out-patient surgical centers by qualified and trained personnel.

The present approach may advantageously address the needed balancing of modulus of elasticity between bone and disc and other anatomical constructs. For example, if using polymethyl methacrylate (PMMA) in bone, which is relatively hard, and a relatively soft “gel” between two bones with PMMA, it is possible that the gel and disc will fail in a particular patient due to an imbalance in materials and choice of anatomic coupling. However, using PMMA in the bone and then a gel in the facet joints instead, the same problem of biomedical balancing of the forces may not be encountered. The imbalance problem may be compounded when attempting to prepare the entire spine or large portions thereof to maintain axial height, as will be appreciated by those skilled in the art.

Other aspects of this approach may include preparation for the procedure including medications for soft tissue relaxation, traction, off loading of the spine to encourage elongation before injection/implantation, and post procedure process to ensure proper mobility of spine. Various configurations of anatomic structures may be used to stiffen, replace, augment, lengthen, shorten, enlarge, and/or contract the patient's spine for the desired outcome. This represents a paradigm shift in that the “normal” group of treatment structures and materials are instead used to proactively maintain spinal height, rather than reactively address the effects of spinal height loss after it occurs which is merely reactive treatment and permits a cascade of deterioration that is less likely to occur in the approaches described herein.

Referring now to FIG. 3, beginning at Block 100, as part of the pre-procedure preparation, the patient provides his/her relevant medical information/history (Block 102) and undergoes a physical examination to measure loads next to bone, disc, and/or facets, at Block 104. Range of motion or excursion is determined for each patient. The relative weight of the head, shoulders, arms and thoracic cavity and contents are determined, as well as the axial load forces on bones and soft tissue components. This may be done by percutaneous devices or implantable “smart” devices to determine the correct combination of structures/materials and locations thereof to achieve desired results for the particular patient. X-ray with motion, MRI, CT and other devices will facilitate more detailed analysis and assist in determination of best combinations or “best fit” products and anatomical parts, as will be appreciated by those skilled in the art.

By way of example, sensors may be implanted along the anatomical structures to determine the motion, forces and loads to the involved structures to plan an appropriate choice of materials and structures for achieving axial height maintenance for the given patient, as will be appreciated by those skilled in the art. The smart device or load cells may be used to determine post-procedure how well forces are balanced and to monitor the patient's progress over time, and any need for modulation or corrective procedure based upon trauma or injury after the procedure. As noted above, not only may “cosmetic” results be achieved, but potentially also improvements in pulmonary, cardiac, and/or metabolic (diabetes, hypertension, osteoporosis) conditions to thereby improve overall health and mobility in some applications, hence more closely matching spinal and medical longevity.

Other important considerations in the procedure are the state of the patient's spine at the time of the procedure. For example, if the procedure is performed in the morning, as opposed to latter in the day, the patient may end up with a different height, as disc height is greatest in the morning after a night of sleep due to increased fluid intake, and decreases with axial loading throughout the day. As such, in some embodiments at least the disc treatment portions of the procedure may be performed in the morning shortly after awakening to facilitate increased height of disc fill, for example. This aspect is further refined by the addition of pre-procedure muscle relaxation with intravenous or intramuscular injection of approved pharmaceuticals, e.g., robaxin, skelaxin, soma, etc. In other embodiments, use of paralyzing agents with general anesthesia may be used for more rigid or stiff patients. Use of SSEP (Somatosensory evoked potential) monitoring may be employed to protect the patient against over distraction or correction, elongation or shortening of the spine resulting in spinal cord injury or other nerve injury.

To this end, traction, bracing, suspension, inversion tables, therapy, muscle relaxants, chiropractic adjustments, etc. may be used to advantageously place the spine in the desired position prior to the procedure, at Block 112. Moreover, medication such as ligament relaxors (e.g., relaxin) may also be used to achieve natural elongation of the spine before the procedures, and after as well, to achieve the desired axial height increase/stability.

Various combinations of traction, stretching, operating room or office machines, tables, and other equipment may be used. In some embodiments it may be beneficial for patients to have manipulation manually of the spine before procedures to increase height of the disc, hydrate the disc, as well as enzymatically or surgically remove parts of a damaged disc or other anatomical components (e.g., bone) in preparation for the procedure.

In addition to such “lengthening procedures,” other areas that may be treated prior to the insertion of stabilizing materials/structure may include facet joints, facet capsule, ligamentum flavum, lamina, transverse process, spinous process, intraspinous and supraspinous ligaments, paravertebral muscle, facia, periosteum, etc. Examples of materials that may be used for the procedure include, for example, PMMA, chymopapain, prostheses, chemical and/or biological materials, energy sources, magnets, etc.

Other aspects of the procedure may include pre-procedure diet, fluid intake, minerals, vitamins, etc. Moreover, pre-procedure physical therapy may also be used to enhance the strength of the specific anatomic components or regions selected. Post procedure, diet, medications, and/or therapy may also be used to enhance the outcome of the procedure, as will be appreciated by those skilled in the art (Block 116). By way of example, a patient may be placed on steroids and/or anti-inflammatories to enhance acceptance of components and to decrease inflammation. Antibiotics may also be used.

Certain exercises may also be used to enhance the structural acceptance of the components and maintain motion, as noted above. Combinations of other therapies such as radio surgery, radiation, chemicals, etc., may be used for certain choices of materials and anatomic structures to prepare them or protect against bone overgrowth, loss of motion or implant failures or needed post procedure modulation, as will be appreciated by those skilled in the art. Here again, devices may also play a role, and implants or external devices may facilitate the determination of post or pre-procedure choice of injection materials or anatomical structures, or post-procedure monitoring of motion, loads and activity of patient to achieve desired goals and tolerance

Stopping loss of axial spine height and maintaining sagital plane balance may improve a patient's sense of well being, lessen depression and/or increase mobility and interaction with society. Moreover, improved medical condition and prevention of fractures and discs and other anatomical component failure may save significant insurance/Medicare costs, and prevent disability from prior art reactive approaches versus the proactive approach set forth herein.

The foregoing will be further understood now with reference to certain patient treatment process examples. Patients will fill out a questionnaire which provides their medical histories (Block 102). From the medical history and physical screening, patients “at risk” for conditions such as osteoporosis and the subsequent fractures resulting therefrom are identified by generating a “score” that is compared to an index to determine what the statistical risk will be for them with respect to which bone(s) is likely to fracture (e.g., T8, L1, etc.), at Block 106. Again, this may be done based upon the fine element model discussed above, for example. From there, it may be determined how many (and which) vertebral bodies, discs, etc. need to be stabilized to prevent such fracturing, if any, at Blocks 108, 110, and 118. By way of example, the index may be based upon variables such as age, life expectancy, race, sex, mobility, medical comorbidities, family history, etc.

The patient may then be informed of the results, proposed treatment, and actions to be taken to help increase the effectiveness of the treatment. As part of the pre-procedure preparation, the patient may be put on a nutrition program, as well as a stretching/traction/physical therapy program to elongate the ligaments and disc. The patient's index findings are matched to the appropriate injections/implants for the bone and other anatomical structures to be treated to provide a substantially balanced modulus of elasticity and at the same time through minimally invasive procedures, maintain full range of motion and activities of daily living. As will be appreciated by those skilled in the art, the selection of implants and materials for a given patient will be based upon engineering principles and characteristics of the products to be used for the specific patient based upon the index, which again may be modeled using the above-described finite element analysis model. Further, as new products become available the index may advantageously be updated to incorporate new product and anatomical construct considerations.

Preferably, the patient may undergo the procedure (Block 114) in the morning, although this need not be the case in all embodiments. However, as noted above, morning is the time when the spine is naturally longer, as opposed to the end of the day, due to the disc height increase without axial load at night. By way of example, the procedure may be performed in a procedure suite/outpatient center under local and possibly general anesthetic with primarily percutaneous and preferably minimally invasive tools, potentially requiring little or no incisions. Post-procedure activities may include stretching, medications to reinforce or enhance the spine preservation and height elongation achieved by the procedure, and physical therapy.

In one exemplary treatment case, a 60 year old woman may be screened for osteoporosis and found to be at risk. She undergoes a DEXA scan and is determined to be osteoporotic and in need of medical treatment to prevent bone loss and fracture. Based upon her index findings, the particular vertebral bodies which are significantly at risk are identified. By way of example, she may have the greatest risk of fracture at the T8 and L1 vertebral bodies. As such, the appropriate treatment for these bones and the surrounding bones, discs, etc., are selected based upon the severity risk of potential fractures, the expected lifespan of the patient, etc. Currently only pharmaceutical treatment is available to her, and she would understand that even with compliance and toleration of the medication and its medical side effects, she would have only a 35-60% reduction in risk of fracture. With a procedural preventative approach as described herein, the reduction in risk of fracture may be further improved depending upon the number of levels addressed.

Moreover, if the patient is not simply concerned with fractures and instead desires a more “aggressive” procedure to help prevent loss of height of the axial spine and prevent kyphosis or a forward bending “old age” appearance, then the index would also be used to select additional implantations for other regions of anatomical stabilization such as facet joints, ligaments and muscle structures to compliment the stabilized bone. The combination of anatomical structures and implant materials/structures may advantageously be balanced and complimentary, and preferably provide minimal invasion at reasonable cost. This more aggressive approach would be done with minimal risk and at potential demonstrative savings to payers compared to the current substantial cost to payers for reactive treatment.

In the example of FIG. 2, one or more types of implants (e.g., PMMA, etc.) are used at a plurality of spaced apart locations along the spine. More particularly, implants are inserted in a region 33 adjacent the cervical and thoracic vertebrae (i.e., C7 and T1), a region 34 adjacent the thoracic and lumbar vertebrae (i.e., T12 and L1), and adjacent the lower lumbar vertebrae (i.e., L5). Again, the number, location, and type of implants used for a particular patient will depend upon the specific parameters of the given patient, and potentially how aggressive the patient wishes to be in adding height and “cosmetic” correction to their appearance.

An exemplary spinoplasty procedure which provides a cosmetic correction for patients with conditions such as dowager humps (which are discussed further below), for example, is now described. In this example, the patient undergoes nearly a percutaneous removal of parts of the cervical and thoracic spinous processes in order to improve the contour and appearance of the posterior aspect of the junction of the neck and upper back. Then the residual shortened spinous processes are fixed together with an implanted structure or element that prevents kyphosis yet allows motion preventing the loss of sagital plane balance of the cervical spine and, at the same time, improving the patient's appearance. In accordance with one embodiment permanent suture material may be used, but those skilled in the art will appreciate that other suitable materials with properties that enhance the desired outcome may also be used.

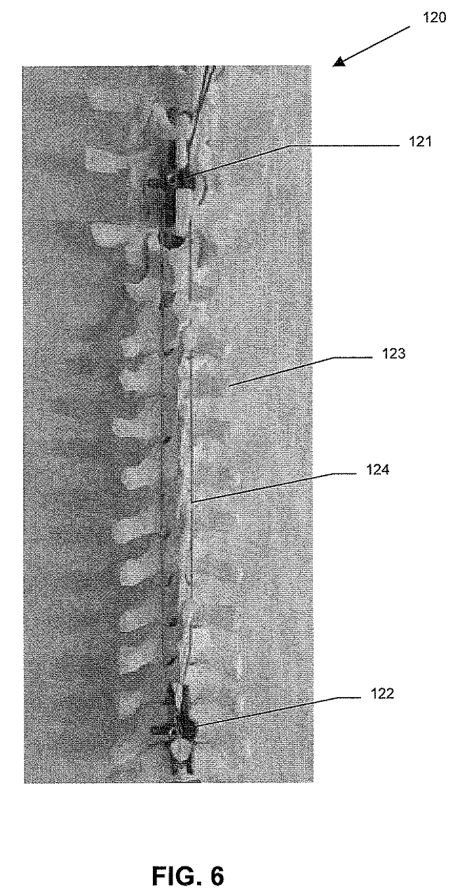

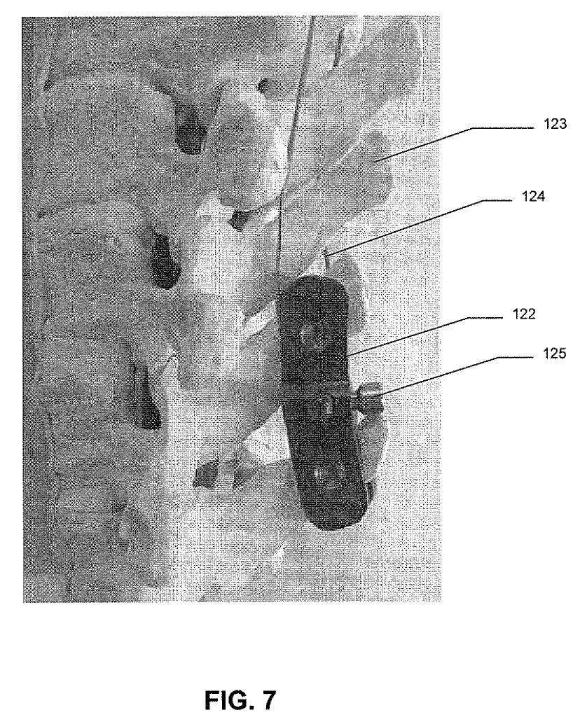

Another exemplary spinal implantation system 120 is shown in FIG. 6. The system 120 includes upper and lower bases 121, 122 that are respectively secured to spinous processes adjacent upper and lower portions of a patient's spine. A cable 124 is connected between the upper and lower bases 121, 122. A tension adjustment screw 125 on the upper or lower bases 121, 122 (or both) may then be used in a posterior percutaneous procedure to adjust the tension on the cable 124 and thereby help prevent the thoracic spine from bending into kyphosis, and also potentially help prevent thoracic compression fractures, for example, as will be appreciated by those skilled in the art.

In accordance with another example, a 70 year old man has a sudden onset of pain in his thoracic region. His doctor orders an MRI or bone scan and determines that he has a pending collapse of a vertebral body as well as osteoporosis. The patient would then enter the treatment program, an index would be determined for him as discussed above, and the appropriate implants/injections would be determined for fracture prevention and/or height stabilization, depending upon the patient's preference. The procedure may be uniquely prepared for the patient's index to help prevent the pending collapse of the particular vertebral bone due to fracture, and if desired to help prevent future loss of axial height.

In still another example, a 50 year old women presents to her physician her concern that her mother and grandmother both became very disabled with very bent spines and pain causing significant loss of mobility, depression and diminished medical health. They both were required to spend their remaining years in nursing home facilities long before their medical health diminished. This patient desires to avoid this cascade of the collapsing spine and bending of the spine with sagital and coronal plane imbalance, which may be likely to occur in her based upon her family history. Here again, an index or model may be used for this patient which will take into account the family history as well as other factors such as DEXA scan results, medical evaluation, etc. The appropriate anatomical structures to be treated and appropriate implant materials/structures therefor are selected accordingly. Again, this would include the appropriate areas of the spine such as: cervical, lumbar and/or thoracic; anterior, posterior, and/or lateral placement, etc.

It should be noted that the above-described procedures may also be used to help prevent the risk of fracture or damage, yet not necessarily prevent spinal axial height loss. In accordance with another example, a 65 year old woman with a history of osteoporosis may desire to prevent fractures. However, she is 6 feet tall and has always been uncomfortable with being taller than her husband. In such case, since she desires to be “less tall,” one or more of the pre-procedure processes intended to lengthen the spine may be omitted. The procedure would then only target the at risk vertebral bodies, and the progressive natural collapse of the disc and other anatomical structures may be allowed to simultaneously prevent loss of sagital plane balance resulting in an “old person look,” but yet allowing controlled decrease in height with age.

In another example, a 45-year-old man, who is 5 feet 4 and has always desired greater height, asks his doctor if there is any way to “safely” be taller. The patient would enter index determination, pre-procedure stretching, traction medical muscle and/or ligament relaxor treatment, etc. After a desired elongation (e.g., 1-2 inches) through this pre-treatment, an early morning procedure may be performed to stabilize the appropriate anatomic structures, namely disc and facet joints, and interspinous ligaments (but not vertebral bodies) to maintain this increased height gain. If at some later point in his life it is determined that he is at risk for fractures, then the vertebral bodies could be subsequently stabilized.

In accordance with a further aspect, in some applications it may be desirable to perform additional procedures prior to one or more of the pre-procedure and/or procedure steps outlined above. By way of example, for cosmetic purposes bone removal, addition and/or spinal contour may first be performed to alleviate an “old person hump” (i.e., a dowager or dowinger hump) on the base of a patient's neck. Thereafter, either with or without the pre-treatment steps described above, the appropriate implants may be selected based upon the index to provide or remove bone and/or height stabilization to prevent further occurrences of humps, as well as to help prevent future fractures and/or maintain axial spinal height.

As will be appreciated by those skilled in the art, a dowagers hump is an abnormal outward curvature of the vertebrae of the upper back. Compression of the front (anterior) portion of the involved vertebrae can lead to forward bending of the spine (i.e., kyphosis), which in turn creates the hump at the upper back. Dowager's hump is typically the result of osteoporotic changes in the thoracic spine, and it may affect both men and women.

In addition to spinal applications, the above-described approach may also be used for other anatomic regions, such as the injection of PMMA or other suitable implant materials/structures to protect the hip femoral neck and shaft from fracturing. Hip fractures are a significant problem with the elderly that often cause severe pain, require invasive surgery with difficult and extended recoveries, and may result in shortening of the leg (and thus loss of height). Another example is to inject PMMA, etc., in the wrist region, as this is presently the third greatest area of fracture after hips and spine. Thus, the proposed approach may provide a procedural-based method to holistically prevent fractures that may otherwise result from osteoporosis, for example.

The hip has ease of percutaneous access through the greater throchanteric region into the neck and shaft. The wrist or distal forearm bones are superficial, and there is ease in percutaneous access as well in this application. This is a paradigm shift to fill a non-fractured wrist bone or hip bone with materials to protect against fractures, and one skilled in the art will recognize many embodiments of percutaneous techniques and products that apply. This substantial paradigm shift for hip and wrists preventative procedural treatment in combination with the spine is a further enhancement of the height preservation techniques set forth herein and matching of the musculoskeletal longevity with medically created longevity.

Currently, significant expenditures are made on medications to help prevent the occurrence of osteoporosis (e.g., bone density enhancing drugs, etc.). Yet, osteoporosis left alone is typically not painful, nor is it known in-and-of-itself to limit quality of life or create other medical issues besides fractures. It is the fractures that case significant pain, suffering, and associated health issues for patients, in addition to a tremendous amount of healthcare dollars for corrective surgeries after the fractures occur. Thus, the “value proposition” in treating osteoporosis through bone density drugs, etc., is the diminution of fractures that are achieved by decreasing bone loss with aging. In other words, if patients did not fracture, medically speaking there would be no reason to treat osteoporosis. Yet, despite the significant expenditure in osteoporosis treatment medicines, a large number of osteoporotic fractures still occur annually (e.g., over 700,000 fractures occurred in 2005 at a cost of over 45 billion dollars for reactive care).

In accordance with a holistic approach, just like the vertebral body pre-fracture or height loss preventative approach, the stabilization of bilateral hip joints, femoral neck and shaft, and/or wrist regions to significantly reduce the overall risk of fractures from falls, etc., in patients with osteoporosis may be achieved, as well as other added benefits such as height maintenance, as discussed above. For hip and wrist treatment, at risk patients may be identified through DEXA scans, bone scans, MRIs, etc. Then using a percutaneous technique such as arcuplasty, PMMA and/or other suitable materials are prophylactically injected into the femoral head, neck, trochanteric region and shaft, and/or around these areas externally to the cortical surface to brace or stabilize these vulnerable areas in the event of a fall or other trauma. By avoiding fractures, the inevitable surgery to correct the fracture, which leads to shortening, may in turn be avoided.

As discussed above, the patient would be evaluated and an index would be used to determine the need for the procedure, as well as the appropriate anatomic regions for treatment and treatment materials/structures. An arcuplasty-type procedure may be used for treating the femur, as will be appreciated by those skilled in the art. More particularly, the patient may have a local or general anesthetic in an out patient center, and the area surrounding the hip joint, etc., would be approached with percutaneous wires, probes, drill, cortical cutters and/or other appropriate tools to enter the area for injection from potentially multiple different possible trajectories (e.g., anterior, posterior, lateral, etc.). The cannula would be advanced into the desired anatomic area at risk in the femoral neck, shaft, head and trochanteric region. PMMA or other suitable substances may be slowly injected to fill the inner aspect of the femur under fluroscopic visualization, for example, to the desired amount to protect the femur from fracture.

In some patients the injected material may be placed externally to the cortical surface and may act as a cushion against falls. Other materials and/or devices may be positioned through minimally invasive surgery techniques to bulk up or protect the vulnerable femor including metals, plastics, polymers, etc., with screws, epoxy or other methods of attachment. This embodiment may utilize not only bony options but also include subcutaneous, fat, bursae and fascia tissues, intervals, compartments and other anatomically available strategies for placing materials to protect against fractures with falls. For example, placing a flexible gortex, plastic, silicone, etc. material inside or outside of the trochanteric bursae may absorb the forces of a direct blow to the femoral neck in a fall, thereby averting fracture. Other bone structures of the body may be treated this way, but the hip is one of the most susceptible to direct forces from falls. This may advantageously and proactively protect the bone against fracture in a preventative fashion, rather than attempting to address damage after a fracture has already occurred.

By way of example, a typical stabilization procedure may take 1-2 hours to perform, depending upon the number of vertebral bodies, discs, etc. that are to receive injections or stabilizing devices. A typical example may include 6-10 levels of vertebral bone, disc, etc., injections, supported with implants or ligamentous structures. In some cases, the spine, hips and wrist (or subset thereof) may be injected at the same time. Times and number of anatomic regions to be treated will of course vary with a given patient's index score and treatment goals, as will be appreciated by those skilled in the art. The procedure would typically involve little or no healing or recovery time, and in many instances may resume regular activities shortly after the procedure.

Another exemplary procedure is for treating an elderly woman with a “dowager hump,” which gives an old age appearance. A 68 year old woman who has maintained a very healthy life style and has stayed youthful in appearance through healthy living and plastic surgery notes that she began to have forward bending of her neck. She has two diagnoses, one for degenerative disc disease (DDD) which caused sagital plane deformity, kyphosis and shortening of the disc spaces accompanied by pain. She also develops a dowager hump and has a protrusion or appearance of a mass at the base of her neck, posteriorly, which she feels is “ugly” and causes her significant vanity issues and concerns for her appearance.

For the first problem (i.e., DDD), due to pain but also appearances, she undergoes an anterior cervical discectomy and fusion (ACOF) to return natural height to the disc, realign her sagital plane and correct the kyphosis. This results in an elongation of her neck and places her head back over her center of gravity with a normal appearance. She also experiences relief of her pain.

For the dowager hump, a spineoplasty procedure is performed. Under local anesthesia, a small incision is made over the bump at the base of the cervical spine. The protruding bone or spinous process is carefully drilled to a smooth contour, which results in a decrease in the bump to a more smooth appearance. Local fatty tissue surrounding the bump is removed. The ligaments are sutured together over the space where the bump was. A material, ligament, tension band or other substance/structure may then be inserted to create the desired appearance of the back of the neck. Using an articulating or artificial disc, it is possible to realign deformity of the spine and in combination with fusion techniques recreate the normal alignment of the cervical spine, preserve axial height and restore balance to maintain axial height and potentially prevent further occurrences of dowager humps.

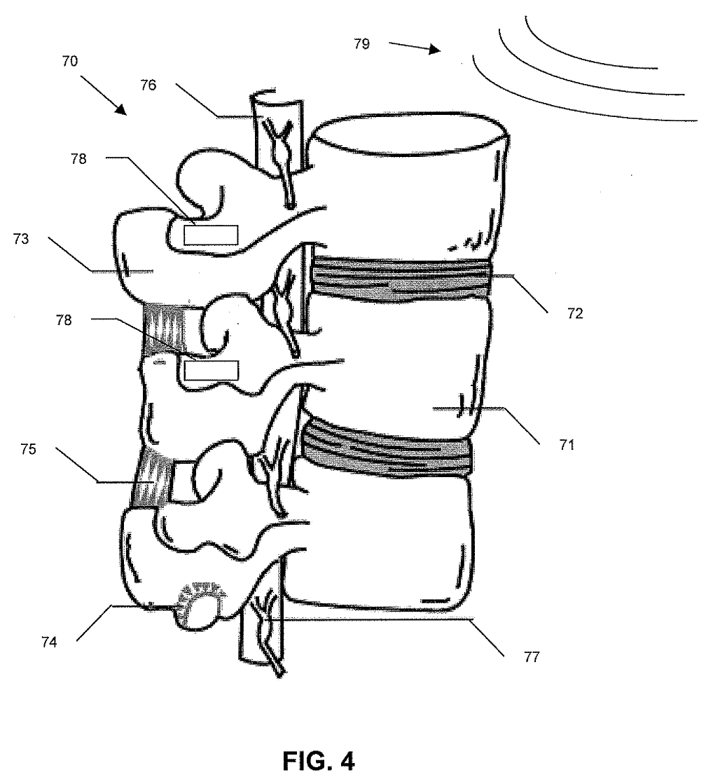

To this end, various types of materials may be used that have properties that can be manipulated percutaneously for adjustment purposes, such as for tightening a ligament or loosening a ligament once it is implanted, as will be appreciated by those skilled in the art. Furthermore, implant materials/structures with properties such as magnetic or chemical slow release that have an effect on the stiffness, relaxation, lengthening, shortening, etc., may also be used. For example, a portion of a spine 70 is shown in FIG. 4 which illustratively includes vertebral bodies 71 with intervertebral discs 72 therebetween. The vertebral bodies 71 include respective spinous processes 73, and ligaments 75 are connected therebetween. The spinal (nerve) column 76 is positioned between the vertebral bodies 71 and spinous processes 73, and spinal nerve roots 77 extend therefrom.

In the illustrated example, metal inserts 78 are implanted in or on the pedicles percutaneously at one or more levels of the spine 70. The metal inserts 78 respond to an external force, electricity, magnets, etc., for manipulation of the spine, elongation, correction of scoliosis or other deformity instead of bracing. For example, this may be done by placing the patient in a magnetic field 79 or providing an electrical current with a machine, body wrap, or bed, chair, etc., for a certain amount of time.

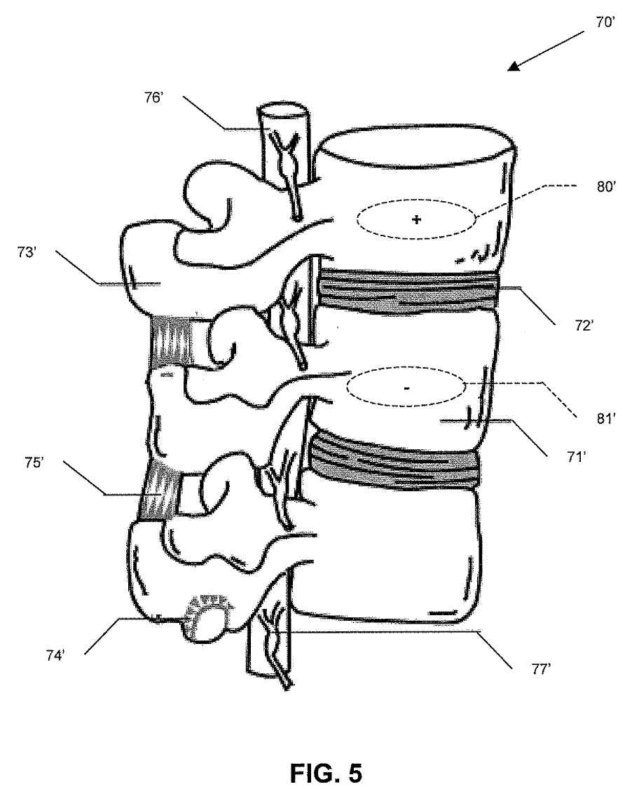

Referring additionally to FIG. 5, the various bony components of the spine may advantageously be magnetized by injecting or implanting regions 80′, 81′ of particles in PMMA or other products to create relative positive and negative charges, respectively. Based upon the method, the configuration and creation of bone magnets may create purposeful forces designed to prevent collapse of disc and bone as well as kyphosis, stenosis and other deformities, as will be appreciated by those skilled in the art.

Similarly, typical injection materials such as PMMA may be augmented or supplemented with (either by mixing or separate injection) particles that allow metallic or magnetic properties which may help resist fractures, or which may allow external forces to repel adjacent vertebral bodies and/or discs with similar injections or products to produce a spine that essentially pushes itself into distraction for height elongation, depending upon the design of the magnets and fields applied. Further, the use of the magnetic bone structures placed in a specific electromagnetic field can induce elongation or shortening, which may be used in preparation for the final method and procedure. This may also facilitate traction for painful disorders of the spine.

Long-distance space travel is planned by NASA, yet there are concerns of severe osteoporosis and fractures in astronauts from extended periods of weightlessness. Magnetic conversion of bone will render bone responsive to electromagnetic force. It is possible that the astronauts could be placed in electrical fields after creating bone magnets. Forces on the bone would be induced by the electrical field according to Wolf's law to potentially prevent loss of bone and increase bone mass.

It should also be noted that the treatment methods set forth herein apply to other medical conditions beyond osteoporosis. For example, in children with scoliosis there is a concavity and a convexity. In adults, they have in kyphosis a concavity and a convexity. If forces are applied to distract in the concavity and compress in the convexity using the above-described techniques, then deformity prevention and/or correction may be possible. Similarly, in some applications these techniques may be applied to cause vertebral bodies to resist each other when bending or when axial forces are applied to thereby preserve disc height, and thus overall axial height.

Another potential condition for which the above-described procedure may advantageously be applied includes spinal stenosis. Spinal stenosis is caused by a cascade of degeneration starting with the disc. The disc loses its ability to hold onto water, and then fragments and tears occur in the annulus of the disc. Next, the mechanical properties of the disc are diminished, leading to a gradual collapse and subsequent diminished height of the disc. This results in loss of height of the disc. Also, the ligaments and ligamentum flavum become buckled and may push into the cauda equina, spinal cord and exiting nerves. Moreover, the neuroformamen may narrow and the nerves may become damaged and painful. If the vertebral bodies were “magnetized” to resist each other, then as the discs degenerate they would not loose their height because the vertebral body magnets would resist moving towards each other. If the disc did not lose height (or loses less height), then spinal stenosis would not occur. If a patient has spinal stenosis, then the vertebral bodies would be turned into magnets and then manipulated into distraction to stretch the disc, neuroforamina and hence indirectly decompress the nerves.

Metallic devices may also be attached in locations where it is desired to create compression, distraction, or manipulation by an external field or force, for example, distraction of the spinous process to decrease stress on discs and relieve pain. For example, a “metallic ligament” may be placed on the posterior spinous process and attached to the bone at different levels and under the influence of the magnetic or electrical field to cause a bending movement to the implant, which may prevent kyphosis or scoliosis. Again, materials or products such as PMMA, biologics (e.g., BMP), etc., may be used, as will be appreciated by those skilled in the art.

Contrary to prior art reactive treatment approaches, the above-described techniques provide a program for maintenance of axial height and sagital plane balance in patients, particularly the elderly. There is currently a significant need for such an approach as many patients are already experiencing longevity due to medical methods, yet only to become debilitated due to failure of the musculoskeletal system to achieve a concomitant longevity. This could perhaps become a crisis as the so-called “baby boomers” continue to age over the next 20-30 years, placing inordinate demands upon Medicare dollars for potentially ineffective reactive treatment, leading to poor quality of life and suffering.

Many modifications and other embodiments of the invention will come to the mind of one skilled in the art having the benefit of the teachings presented in the foregoing descriptions and the associated drawings. Therefore, it is understood that the invention is not to be limited to the specific embodiments disclosed, and that modifications and embodiments are intended to be included within the scope of the appended claims.

That which is claimed is:

1. A proactive spinal treatment method for maintaining axial spine height and sagital plane spine balance of a spine comprising vertebrae and intervertebral discs between adjacent vertebrae, the method comprising:
collecting a plurality of spinal health parameters relating to predicted spinal degeneration risk for a given patient's spine;
analyzing the plurality of spinal health parameters to generate a proposed stabilizing implantation treatment using a finite element spinal model; and
performing the proposed stabilizing implantation treatment on the given patient's spine to proactively treat the patient to maintain axial spine height and sagital plane spine balance.

2. The method of claim 1 wherein performing the proposed stabilizing implantation treatment comprises implanting at least one stabilizer adjacent a plurality of spaced apart locations along the spine.

3. The method of claim 1 wherein the plurality of spinal health parameters are selected from a group comprising patient age, medical comorbidities, family history, and patient fracture history.

4. The method of claim 1 wherein the plurality of spinal health parameters are selected from a group comprising dual energy x-ray absorptiometry (DEXA) scan results, magnetic resonance imaging scan results, and computerized axial tomography scan results.

5. The method of claim 1 wherein performing the proposed stabilizing implantation treatment comprises implanting a plurality of opposing magnetic elements within the given patient's spine.

6. The method of claim 6 wherein implanting the plurality of opposing magnetic elements comprises implanting opposing regions of polymethylmethacrylate (PMMA) comprising magnetic particles of opposite polarity.

7. The method of claim 1 wherein performing the proposed stabilizing implantation treatment comprises implanting at least one metallic element between an opposing pair of vertebrae and inducing a magnetic field for causing the at least one metallic element to space apart the pair of vertebrae.

8. The method of claim 1 further comprising performing a spinal elongation procedure to elongate the given patient's spine to an elongated state before performing the proposed stabilizing implantation treatment.

9. The method of claim 8 wherein the spinal elongation procedure comprises at least one of traction, bracing, suspension, inversion, and chiropractic manipulation.

10. A proactive spinal treatment method for maintaining axial spine height and sagital plane spine balance of a spine comprising vertebrae and intervertebral discs between adjacent vertebrae, the method comprising:
collecting a plurality of spinal health parameters relating to predicted spinal degeneration risk for a given patient's spine; and
analyzing the plurality of spinal health parameters to generate a proposed stabilizing implantation treatment using a finite element spinal model, the proposed stabilizing implantation treatment to be performed on the given patient's spine to proactively treat the patient to maintain axial spine height and sagital plane spine balance.

11. The method of claim 10 wherein the plurality of spinal health parameters are selected from a group comprising patient age, medical comorbidities, family history, and patient fracture history.

12. The method of claim 10 wherein the plurality of spinal health parameters are selected from a group comprising dual energy x-ray absorptiometry (DEXA) scan results, magnetic resonance imaging scan results, and computerized axial tomography scan results.

13. A proactive spinal treatment method for maintaining axial spine height and sagital plane spine balance of a spine comprising vertebrae and intervertebral discs between adjacent vertebrae, the method comprising:
collecting a plurality of spinal health parameters relating to predicted spinal degeneration risk for a given patient's spine;
analyzing the plurality of spinal health parameters to generate a proposed stabilizing implantation treatment using a finite element spinal model and comprising implantation of at least one stabilizer adjacent a plurality of spaced apart locations along the spine;
performing a spinal elongation procedure to elongate the given patient's spine to an elongated state; and
performing the proposed stabilizing implantation treatment on the given patient's spine to proactively treat the patient to maintain axial spine height and sagital plane spine balance.

14. The method of claim 13 wherein the plurality of spinal health parameters are selected from a group comprising patient age, medical comorbidities, family history, and patient fracture history.

15. The method of claim 13 wherein the plurality of spinal health parameters are selected from a group comprising dual energy x-ray absorptiometry (DEXA) scan results, magnetic resonance imaging scan results, and computerized axial tomography scan results.

16. The method of claim 13 wherein performing the proposed stabilizing implantation treatment comprises implanting a plurality of opposing magnetic elements within the given patient's spine.

17. The method of claim 16 wherein implanting the plurality of opposing magnetic elements comprises implanting opposing regions of polymethylmethacrylate (PMMA) comprising magnetic particles of opposite polarity.

18. The method of claim 13 wherein performing the proposed stabilizing implantation treatment comprises implanting at least one metallic element between an opposing pair of vertebrae and inducing a magnetic field for causing the at least one metallic element to space apart the pair of vertebrae.

19. The method of claim 13 wherein the spinal elongation procedure comprises at least one of traction, bracing, suspension, inversion, and chiropractic manipulation.
